(19) United States
(12) Patent Application Publication
(10) Pub. No.: US 2007/0225819 A1
(43) Pub. Date: Sep. 27, 2007

(54) Apparatus and method for the treatment of periprosthetic fractures

(75) Inventor: David T. Eva, Seattle, WA (US)

Correspondence Address: GRAYBEAL, JACKSON, HALEY LLP, 155 - 108TH AVENUE NE, SUITE 350, BELLEVUE, WA, 98004-5973, US

(73) Assignee: DePuy Products, Inc., Warsaw, IN (US)

(21) Appl. No.: 11/388,263

(22) Filed: Mar. 24, 2006

(51) Int. Cl. A61F 2/38

(52) U.S. Cl. 623/20.14

(57) ABSTRACT

An apparatus and method is provided for the reduction and fixation of fractures of a bone to which an implant component has been attached. The apparatus includes a reduction plate that is configured to be anchored to the implant component and to receive bone engaging fasteners to fasten the reduction plate to the bone spanning the fracture. The implant component is provided with attachment points to which the reduction plate may be anchored to the component if and when a fracture occurs. The apparatus may also include an intramedullary nail configured to be anchored to the implant component.

### BACKGROUND OF THE INVENTION

The present invention relates to orthopaedic implants, and particularly to joint prostheses. More specifically, the invention provides an apparatus and method for the treatment of fractures of bones supporting implant components.

Over the last few decades, orthopaedic implants for the replacement of damaged or diseased joints has become more prevalent. For example, by the late 1990's, the number of total knee arthroplasties performed in the United States rose to about 150,000 per year. That number is expected to increase each year as the average age of the population increases. Of course, the number of knee and other joint arthroplasties will be much greater world-wide.

As the number of implants placed in patients increases, it is inevitable that associated fractures also become more common. In some cases, the implant itself increases the likelihood of a periprosthetic fracture due to osteolysis and stress risers associated with osteolytic bone defects. In other cases, the medical condition that necessitated the arthroplasty still exists and eventually leads to a local fracture. For example, the rate of supracondylar fractures after total knee replacement has been found to approach 2.5%. Common problems associated with periprosthetic fractures includes mal-alignment, stiffness and non-union. If mal-alignment occurs, the abnormal joint mechanics may lead to a high rate of revision secondary to implant loosening.

The presence of the implant often makes treatment of periprosthetic fractures very difficult. In some cases, the presence of the implant may impair fracture healing because of endosteal ischemia. It has been observed that rates of non-union for supracondylar fractures proximal to a total knee prosthesis are higher than for the same fractures in the absence of an implant. Stems, rods, screws and methylmethacrylate may block the medullary canal, preventing intramedullary fixation of a fracture.

Of course, the treatment of periprosthetic fractures must adhere to the basic principles of treating any fracture. In particular, the biomechanical integrity of the bone must be restored, which means the restoration of a biological environment in which the bone can heal and a mechanically stable construct to give the bone a chance to heal. Mechanical stability is obtained by providing adequate fixation distal and proximal to the fracture. Most periprosthetic fractures require surgical stabilization using selected implants.

Depending upon the location of the fracture, implant options include flexible intramedullary rods, rigid intramedullary rods, bone plates and cerclage wires. Certain intramedullary rods incorporate anchoring features, such as the Zickel intramedullary rod that is anchored by transverse nails through openings in the rod. For femoral fractures above a total knee replacement, one typical treatment includes placement of antegrade nails.

Plate fixation is appropriate for some long bone fractures. However, one problem with fixation plates is that they require sufficient purchase in the bone to fully stabilize the fracture and fix the plate. This problem is particularly acute for reduction and fixation of fractures immediately adjacent a joint prosthesis, such as periprosthetic fractures of the distal femur.

There is a need for a device that can readily and efficiently reduce and fix a periprosthetic fracture.

### SUMMARY OF THE INVENTION

To address this need, the present invention provides an apparatus and method for the reduction and fixation of fractures of a bone to which an implant component has been attached. The apparatus includes a reduction plate that is configured to be anchored to the implant component and to receive bone engaging fasteners to fasten the reduction plate to the bone spanning the fracture. The implant component is provided with attachment points to which the reduction plate may be anchored to the component if and when a fracture occurs.

In one embodiment, the reduction plate includes a base that defines fixation holes to receive set screws. The set screws are configured to engage threaded anchor bores in the implant component. The reduction plate further includes a fixation element, which in the preferred embodiment is an elongated plate having a plurality of fixation openings defined therethrough. The fixation openings are configured to receive bone engaging fasteners, which are preferably bone screws suitable for reduction and fixation of bone fractures. The reduction plate is sized to extend from an anchor point on the implant component and span the fracture. The plurality of openings provides a selection of locations for fixation of the fracture with the bone screws.

In a further embodiment, the apparatus incorporates an intramedullary nail. In this embodiment, the implant component includes a tapered opening in a portion of the component that is aligned with the intramedullary canal of the bone when the implant component is properly positioned. The intramedullary nail includes a shank adapted to extend into the intramedullary canal and head with a tapered outer surface. The outer surface is adapted to form a solid press-fit engagement with the tapered opening in the implant component.

In accordance with one method of the invention, an implant component is provided with anchor points on a wall of the component that is exposed and accessible after the implant component has been attached to the bone. In the case of a femoral component of a total knee prosthesis, the anchor points may include threaded bores in the side walls of the implant component. The anchor points do not interfere with the implantation or operation of the prosthesis and may never be used. However, if a fracture occurs adjacent the implant component, the component is surgically accessed, along with the fractured portion of the bone. The reduction plate may be preliminarily positioned to determine whether the plate needs to be contoured to match the bone and to evaluate the location of the bone fasteners.

Once that determination has been made, the reduction plate may be anchored to the implant component, such as by the use of set screws. The bone screws are then driven into the bone through the fixation openings in the reduction plate, thereby fixing the fracture and attaching the reduction plate to the bone. The placement and fixation of the bone screws and reduction plate may be accomplished in a conventional manner, which should be unchanged by anchoring the base of the reduction plate to the implant component.

It is one object of the invention to provide an apparatus and method that permits stable reduction and fixation of periprosthetic fractures. It is a further object to provide an apparatus that can be anchored to a well-fixed implant component, but only when necessary to fix a fracture. Other objects and certain benefits of the invention will become apparent upon consideration of the following written description and accompanying figures.

### DESCRIPTION OF THE FIGURES

FIG. 1 is a perspective view of a femoral component of a knee prosthesis with an apparatus for fixation of a femoral fracture in accordance with one embodiment of the present invention.

FIG. 2 is a perspective view of the femoral component of a knee prosthesis modified in accordance with one aspect of the invention.

FIG. 3 is a top elevational view of a reduction plate for use with the femoral component shown in FIG. 2 to form the apparatus for fixation illustrated in FIG. 1.

FIG. 4 is a side elevational view of the reduction plate shown in FIG. 3.

FIG. 5 is a side elevation al view of a reduction plate in an alternative embodiment for use with the femoral component shown in FIG. 2.

FIG. 6 is perspective view of a femoral component of a knee prosthesis modified in accordance with another embodiment of the invention to fix an intramedullary nail within the intramedullary canal of the femur.

FIG. 7 is a perspective partial sectional view of the femur with the femoral component and intramedullary nail shown in FIG. 6 fixed to the bone.

### DESCRIPTION OF THE PREFERRED EMBODIMENTS

For the purposes of promoting an understanding of the principles of the invention, reference will now be made to the embodiments illustrated in the drawings and described in the following written specification. It is understood that no limitation to the scope of the invention is thereby intended. It is further understood that the present invention includes any alterations and modifications to the illustrated embodiments and includes further applications of the principles of the invention as would normally occur to one skilled in the art to which this invention pertains.

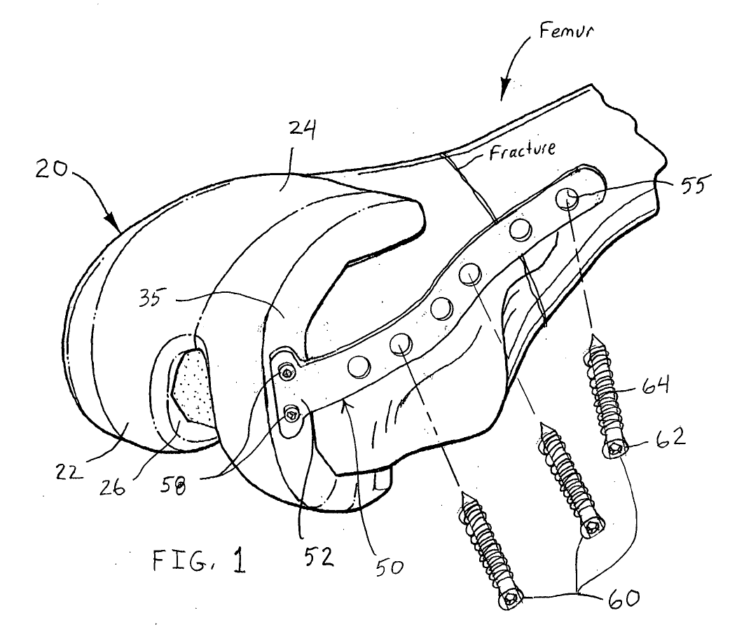

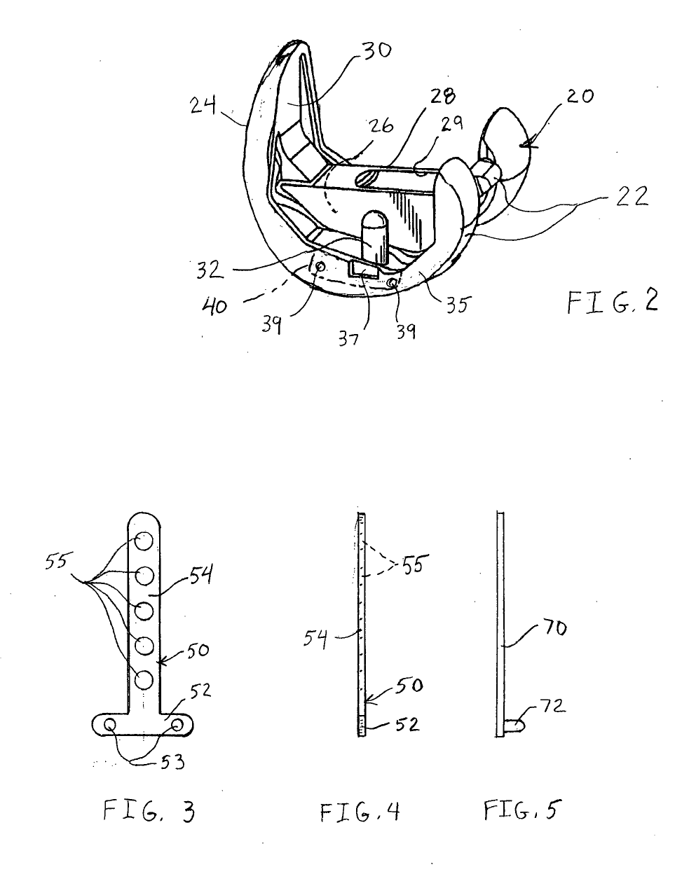

The present invention concerns an apparatus and method for reduction and/or fixation of a periprosthetic fracture—i.e., a fracture of a bone in which a prosthetic joint component has been implanted. In the illustrated embodiment, the joint is the knee and the fracture is in the distal femur, adjacent the femoral component of the prosthetic knee. Thus, as shown in FIG. 1, a femoral component 20 is fixed to the distal end of the femur. The femoral component may be of a variety of configurations, such as the femoral component disclosed in U.S. Pat. No. 6,926,738, the disclosure of which is incorporated herein by reference. For example, the component 20 may include an anterior bearing 24 and condyle bearings 22 separated by an intercondylar notch 26, as depicted in FIGS. 1 and 2. The component 20 may further include a box 28 with an aperture 29, in which the box surrounds the intercondylar notch 26 and is configured to engage a stabilizing post of the proximal tibial component, as disclosed in the '738 Patent. The component 20 includes a bone engaging surface 30 that contacts the prepared end of the femur or provides a bone cement interface. Fixation posts 32 may also be provided to enhance fixation of the component to the femur. A tool notch 37 may be incorporated into each side wall 35 of the implant component to facilitate placement of the component 20 on the distal end of the femur.

As thus far described, the femoral component is configured and operates as part of the joint prosthesis disclosed in the '738 Patent, or any other known femoral component. The femoral component 20 is implanted in accordance with known procedures and in most cases the patient experiences no significant complications or difficulties with the implant. However, for some of the reasons set forth above, some patients may suffer a fracture near the prosthesis, as depicted in FIG. 1. Like any fracture, periprosthetic fractures require adequate reduction and solid fixation for the femur to mend. Thus, the present invention contemplates a fracture reduction plate 50 that is engaged to the implant component 20 to provide a solid base for reduction of the fracture and fixation of the bone. As shown in FIGS. 3-4, the reduction plate 50 may be generally T-shaped with a base 52 and a fixation element 54 extending therefrom. In the preferred embodiment, the fixation element is an elongated plate integral with the base 52. The element defines a plurality of openings 55 adapted to receive and support a bone engaging fastener. In one embodiment, the fasteners are bone screws 60, as shown in FIG. 1. The bone screws 60 include an enlarged head 62 that may be configured to nest within a corresponding opening 55. The shank 64 of the may be provided with appropriate threads for fixation within the affected bone. Alternatively, the fasteners may take other forms suitable for reduction and fixation of a fractured bone, with commensurate modifications to the plate 54 and openings 55 to receive the fasteners.

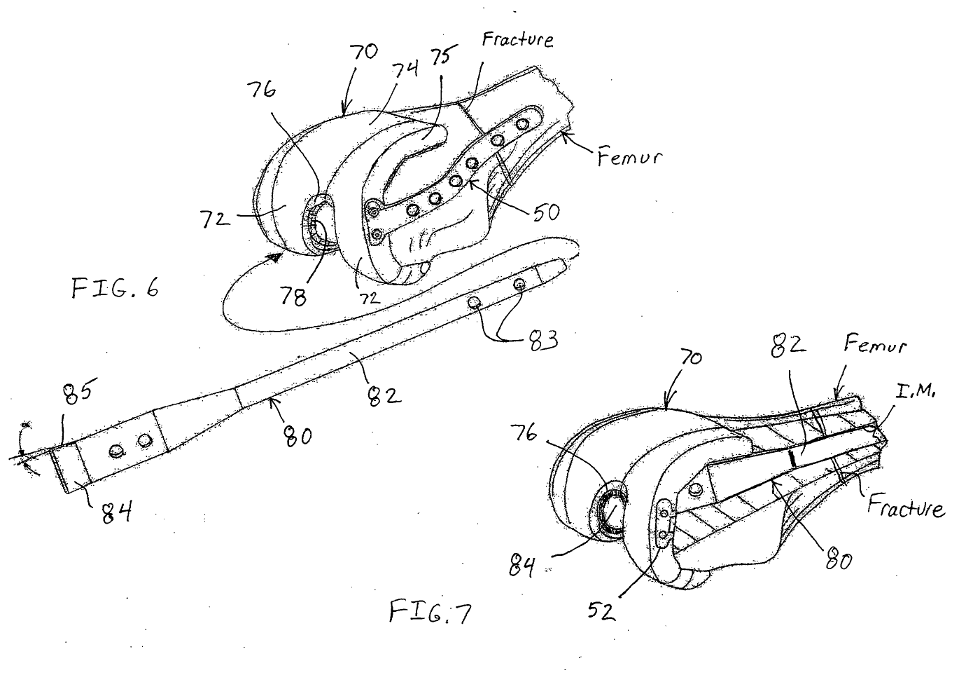

The femoral implant component 20 is configured to serve as an anchor for the base 52 of the reduction plate 50. In particular, the side walls 35 of the component 20 may be provided with anchor bores 39 that correspond to holes 53 defined in the base 52 of the plate. In one embodiment, the bores 39 are threaded to receive a set screw 58 (FIG. 1) that passes through the holes 53 to mount the plate to the implant component. Alternatively, the bores 39 in the component side walls 35 may be tapered bores, such as at a Morse taper angle, to receive a press-fit tapered fastener passing through the holes 53. In yet another alternative, the holes 53 in the plate base 52 may also be threaded to accept a fastener having threads adjacent the head of the fastener. In yet another alternative, the reduction plate, such as plate 70 in FIG. 5, can be provided with integral tapered posts 72 adapted to be press-fit into a tapered anchor bore 39. It is contemplated that any suitable fastener and attachment interface may be used to attach a reduction plate 50 to one or both of the side walls 35 of the implant component 20. In the illustrated embodiment, two attachment points are provided on the base 52, with the mating attachment points (i.e., bores 39) flanking the positioning tool notch 37. However, other numbers and positions of attachment points are contemplated based on the available space on the side walls 35 of the implant component 20.

In one embodiment, the base 52 of the reduction plate 50 is fixed against the surface of the side wall 35. In an alternative, the side walls 35 may be provided with an indentation 40 centered around the anchor bores 39, as depicted in phantom lines in FIG. 2, which conforms to the perimeter of the base 52. The depth of the indentation is preferably less than or equal to the thickness of the reduction plate 50. The indentation can help locate and stabilize the reduction plate 50 when it is attached to the implant component.

In the embodiment illustrated in FIG. 3, the reduction plate 50 is essentially T-shaped. However, other orientations and configurations of the fixation element 54 are also contemplated. For instance, the element 54 may be an elongated plate situated at a non-perpendicular angle relative to the base 52. The fixation element 54 may also have a shape other than the elongated shape shown in FIG. 3, with that shape being dictated by the geometry of the bone that is to be reduced and fixed. It is understood that the purpose of the fixation element is to provide sufficient points of attachment to the bone to allow use of the bone fasteners 60 where they can obtain adequate purchase and are properly positioned to help reduce the fracture. The fixation element 54 may be further configured to accept adjunct fixation components, such as cerclage wires. In the preferred embodiment, the fixation element 54 may be bent or contoured to solidly contact the bone, as depicted in FIG. 1. The ability to contour the element 54 is determined in part by the material and in part by the thickness of the plate. In the preferred embodiment, the plate is formed of a medical grade stainless steel with a thickness of about 1-5 mm.

In accordance with a method of the present invention, an implant component having anchor bores defined in an accessible wall of the component is implanted as part of a joint arthroplasty procedure. The implant component may, for instance, be a femoral component, such as the component 20 of the illustrated embodiment, having the anchor bores 39 defined in both side walls 35 of the component. The component is implanted and fixed to the bone in a conventional manner. The exposed anchor bores may be capped with a press-fit removable cap or filled with a non-hardening putty to prevent tissue ingrowth into the bores. The anchor bores 39 nominally play no part in the arthroplasty procedure, although they may be used to mate with an appropriately configured insertion tool.

For most patients, the anchor bores will never need to be used because no periprosthetic fracture will occur. However, in the event of such a fracture, the implant component can then serve to anchor the reduction plate 50 of the present invention to help reduce and fix the fracture. The same surgical procedure may be used with the reduction plate 50 as would be used to fix the fracture with stand-alone plates or fracture fixation devices. The side walls 35 of the implant component are accessed and the anchor bores 39 cleared and prepared for attachment of the reduction plate 50.

The reduction plate 50 may then be positioned with the attachment holes 53 lined up with the anchor bores 39. This position can be held temporarily with pins loosely fitted through the holes and into the bores. This temporary positioning of the reduction plate 50 is to allow the surgeon to determine whether the plate needs to be contoured. This temporary positioning can also help identify where the bone engaging fasteners, such as the screws 60, can be placed to reduce and fix the fracture. The fixation openings 55 can be used to help position a drill guide or K-wire so that the bone can be prepared to receive the fastener. In addition, the temporary positioning can be used to determine the necessary length of the fixation element 54. It is contemplated that a range of sizes or lengths of the reduction plate 50 is provided from which the surgeon can select the optimally sized plate to be implanted.

Once the bone has been prepared to receive the fasteners, the reduction plate 50 is mounted to the well-fixed implant component. In one embodiment, the plate 50 is mounted by driving set screws 58 through the openings 53 into the threaded anchor bores 39 of the component 20. The bone fasteners 60 are then driven through the openings 55 into the prepared locations in the bone as the fracture is reduced. Depending upon the nature of the fracture, the same process may be conducted with a reduction plate 50 on the opposite side wall of the implant component so that reduction plates are used on opposite sides of the bone to reduce and fix the fracture.

In another embodiment of the invention, an implant component is adapted to anchor an intramedullary nail, as illustrated in FIGS. 6-7. In this embodiment, a femoral component 70 is fixed to the femur. The component 70 includes condyle bearings 72, an anterior bearing surface 74 and an intercondylar notch 76. Unlike the femoral component 20 in the previous embodiment, the femoral component 70 is not configured to mate with a stabilizing element on the tibial portion of the joint prosthesis. Thus, the intercondylar notch 76 of this embodiment does not incorporate the box 28 of the component 20 (FIG. 2). Instead, in this embodiment of the invention, the implant component 70 is provided with a bore 78 defined within the intercondylar notch 76. The bore 78 is provided with an inwardly tapered wall. In accordance with the invention, this bore is arranged in the implant 70 so that it will substantially align with the intramedullary (I.M.) canal of the bone when the implant component is properly installed on the bone.

The apparatus of this embodiment further includes an intramedullary nail 80 with a shank 82 sized to fit within the intramedullary canal of the bone, such as the femur. As illustrated in FIG. 7, the shank 82 has a length sufficient to traverse the fracture. This shank portion of the nail may have any known configuration in the art for the affected bone.

The intramedullary nail 80 includes an enlarged head 84 having a tapered outer surface 85. The tapered surface 85 is adapted to form a solid press-fit engagement with the tapered bore 78 in the implant component 70. In a specific embodiment, the bore and surface may be defined at a Morse taper angle. As shown in FIG. 7, the nail 80 is driven into the intramedullary (I.M.) canal of the bone and the head 84 is driven into the tapered bore 78 at the intercondylar notch 76 of the implant component. The nail 80 is thus anchored to the implant component. As shown in FIG. 6, the nail 80 may be used in lieu of the reduction plate 50, if the plate is present on the implant component. However, in certain circumstances, it may be desirable to combine the two forms of fixation—i.e., the reduction plate 50 and nail 80. In certain specific embodiments, the nail may be provided with openings 83 along the shank 82 that are nominally intended for bone ingrowth, but that may serve to receive a bone screw aligned with an opening in the reduction plate.

The present invention contemplates an adjunct to an existing implant component that facilitates treatment of periprosthetic fractures. In particular, the reduction plate of the present invention uses the well-fixed implant component as a base to help reduce the fracture. Since the reduction plate is attached to the implant component, the fixation of the reduction plate itself is more stable. Moreover, since the eduction plate is anchored to the implant, it can provide access to viable bone adjacent the implant that may not otherwise be accessible to separate and independent fixation plate systems. While the implant component 20 in the illustrated embodiment is a femoral component and the fracture is of the distal femur, or implants and bones may be treated with the present invention. For instance, the implant component may be the tibial component of a total knee prosthesis and the fracture bone the tibia. In this case, the reduction plate 50 may be configured for attachment to the tibial plate of the tibial component.

While the invention has been illustrated and described in detail in the drawings and foregoing description, the same should be considered as illustrative and not restrictive in character. It is understood that only the preferred embodiments have been presented and that all changes, modifications and further applications that come within the spirit of the invention are desired to be protected.

For instance, the fixation plate 54 may be provided with a bone engaging surface, such as small spikes protruding from the facing surface of the plate. In another alternative, the attachment holes 53 may be in the form of slots to permit variable positioning of the fastener 60 relative to the slot. The addition of slots may permit use of the bone engaging fasteners to help reduce the fracture before the fasteners are finally tightened to the fixation plate 54. In still another variation, at least some of the fasteners may be bone bolts that are fixed within the bone before the reduction plate is introduced. The bone bolts would incorporate a threaded shank that extends through the attachment openings 55 for engagement with a nut to attach the plate 50 to the fastener.

It is further contemplated that the base 52 of the reduction plate 50 may be configured to permit attachment of the plate to existing attachment points on an implant component. For instance, certain implants may include threaded bores used to engage an implant tool. If those threaded bores are accessible once the prosthesis component has been implanted, then those attachment points may be used to anchor the reduction plate. Appropriate modification to the base 52 of the plate 50 may be necessary to match the geometry of the prosthesis component at the attachment points.

What is claimed is:

1. An apparatus for use with a component of an implant attached to a bone to treat a fracture of the bone comprising:
a fixation element configured for placement on the bone across the fracture;
a base connected to the fixation element and configured to engage the implant component; and
at least one bone engaging fastener having a portion configured to engage the bone and a portion configured to engage the fixation element.

2. The apparatus of claim 1, wherein said fixation element includes an elongated plate defining a plurality of openings for receiving said at least one bone engaging fastener therethrough.

3. The apparatus of claim 2, wherein said at least one bone engaging fastener is a bone screw having a shank configured to extend through one of said plurality of openings and including bone engaging threads for fixation within the bone and a head configured to engage said elongated plate.

4. The apparatus of claim 1, wherein said base defines at least one hole therethrough and includes a fastener configured to pass through said at least one hole to engage the implant component.

5. The apparatus of claim 4 in which the implant component includes at least one wall that is exposed and accessible after the component has been attached to the bone, and the wall defines at least one threaded bore, wherein said fastener is a set screw configured to threadedly engage the threaded bore in the implant component.

6. The apparatus of claim 4 in which the implant component includes at least one wall that is exposed and accessible after the component has been attached to the bone, and the wall defines at least one tapered bore, wherein said fastener is configured for press-fit engagement within the tapered bore in the implant component.

7. The apparatus of claim 1, wherein said fixation element is contourable.

8. An apparatus for treating a bone fracture comprising:
an implant component fastenable to the bone, said implant component including at least one wall that is exposed and accessible after the component has been fastened to the bone, said at least one wall defining at least one anchor bore therein;
a reduction plate including;
a base defining at least one opening therethrough corresponding to said at least one anchor bore;
a fastener configured to extend through said at least one opening to engage said at least one anchor bore; and
a fixation element connected to said base and configured for placement on the bone across the fracture, said fixation element defining a plurality of fixation openings therethrough; and
at least one bone engaging fastener having a portion configured to extend through one of said plurality of fixation openings to engage the bone and a portion configured to engage the fixation element.

9. The apparatus of claim 8, wherein:
said anchor bore is threaded; and
said fastener is a set screw configured to threadedly engage the threaded anchor bore.

10. The apparatus of claim 8, wherein:
said anchor bore is tapered; and
said fastener is configured for press-fit engagement within the tapered anchor bore.

11. The apparatus of claim 8, wherein said fixation element is contourable.

12. The apparatus of claim 8, wherein said fixation element is an elongated plate.

13. The apparatus of claim 8, wherein said at least one bone engaging fastener is a bone screw having a shank configured to extend through one of said plurality of openings and including bone engaging threads for fixation within the bone and a head configured to engage said elongated plate.

14. A method for treatment of a fracture of a bone having an implant component fastened thereto comprising:
fixing a reduction plate across the fracture; and
anchoring the reduction plate to the implant component.

15. The method of claim 14, wherein the step of anchoring the reduction plate includes:
providing an implant component having at least one anchor bore defined on a wall of the component that is accessible when the component is fastened to the bone; and
engaging a fastener between the reduction plate and the anchor bore to anchor the reduction plate to the implant component.

16. The method of claim 14, wherein the step of fixing a reduction plate includes contouring at least a portion of the reduction plate to contact the bone.

17. The method of claim 14, wherein the step of fixing a reduction plate includes:
providing a reduction plate having at least one fixation opening;
positioning the reduction plate against the bone; and
driving a bone engaging fastener through the fixation opening into the bone.

18. The method of claim 17, wherein the step of fixing the reduction plate includes:
providing a reduction plate having a plurality of fixation openings; and
driving a bone engaging fastener through selected ones of the fixation opening into the bone.

19. An implant component forming part of a prosthetic joint comprising:
an implant body configured for attachment to a portion of a bone at a joint; and
a fracture fixation element having a bone engaging portion and an implant engaging portion independent of the attachment of the implant body to the bone.

20. The implant component of claim 19, wherein said fracture fixation element includes an elongated plate having a base configured to engage said implant body and defining a plurality of openings for receiving a bone engaging fastener therethrough.

21. The implant component of claim 20, further comprising at least two bone engaging fasteners having a portion configured to engage the bone and a portion configured to engage said plate at a corresponding one of said openings.

22. The implant component of claim 20, wherein:
said implant body includes at least one wall that is exposed and accessible when the body is attached to the bone, said at least one wall defining at least one threaded bore;
said base of said elongated plate defines at least one hole corresponding to said at least one threaded bore; and
the implant component further includes a set screw configure to pass through said at least one hole and into threaded engagement with said corresponding threaded bore to anchor said elongated plate to said implant body.

23. The implant component of claim 19, wherein said fracture fixation element is an intramedullary nail in which said bone engaging portion is an elongated shank of said nail and said implant engaging portion is an enlarged head.

24. The implant component of claim 23, wherein said implant body defines a bore sized to receive said enlarged head of said intramedullary nail therein, said bore arranged to align with the intramedullary canal of the bone when said implant body is attached to the bone.

25. The implant component of claim 24, wherein said enlarged head and said bore in said implant body define a press-fit engagement.

26. The implant component of claim 25, wherein said press-fit engagement includes complementary tapered surfaces on said enlarged head and in said bore.

27. The implant component of claim 24, wherein:
said implant body is configured as a femoral component for a knee prosthesis, said body defining a intercondylar notch between condyle bearing surfaces, said bore defined within said intercondylar notch.

28. The implant component of claim 23 further comprising:
an elongated plate having a base configured to engage said implant body and defining a plurality of openings; and
at least two bone engaging fasteners having a portion configured to engage the bone and a portion configured to engage said plate at a corresponding one of said openings.
